(19) United States
(12) Patent Application Publication
(10) Pub. No.: US 2020/0394491 A1
(43) Pub. Date: Dec. 17, 2020

(54) METHODS FOR SEQUENCING BIOMOLECULES

(71) Applicant: KONINKLIJKE PHILIPS N.V., EINDHOVEN (NL)

(72) Inventors: Yee Him Cheung, Boston, MA (US); Nevenka Dimitrova, Pelham Manor, NY (US); Balaji Srinivasan Santhanam, Elmsford, NY (US)

(21) Appl. No.: 16/638,532

(22) PCT Filed: Aug. 13, 2018
(86) PCT No.: PCT/EP2018/071861

§ 371 Date: Feb. 12, 2020

Related U.S. Application Data
(60) Provisional application No. 62/547,337, filed on Aug. 18, 2017.

(51) Int. Cl. G06N 3/00; G16B 25/10; G16B 30/00; G01N 33/68; C12Q 1/6869; G06F 17/15

(52) U.S. Cl. CPC G06N 3/002; G16B 25/10; G06F 17/15; G01N 33/6818; C12Q 1/6869; G16B 30/00

(57) ABSTRACT

A system and method for providing sequencing of biomolecules, which can be used for differential analysis of a test sample from a normal sample. Methods can involve steps of providing a mapped sequence file of each of a pilot test sample and a pilot normal sample, wherein each sequence file has a pilot number of reads; calculating, by a processor, a first test-normal genomic comparison pilot view from the sequence files of the pilot test sample and the pilot normal sample, wherein the first pilot view distinguishes pilot test sample data from pilot normal sample data based on at least one genomic parameter; calculating, by the processor, for each sequence file a downsampled sequence file having a reduced pilot number of reads; calculating, by the processor, a second test-normal genomic comparison pilot view from the downsampled sequence files of the pilot test sample and the pilot normal sample, wherein the second pilot view distinguishes the pilot test sample data from the pilot normal sample data based on the at least one genomic parameter; repeating the downsampling steps for determining the fewest pilot number of reads required for calculating a test-normal genomic comparison view that distinguishes the pilot test sample data from the pilot normal sample data based on the at least one genomic parameter; sequencing biomolecules of the test sample and the normal sample using a number of reads equal to the fewest pilot number of reads; calculating, by the processor, a test-normal genomic comparison view for displaying the differential analysis based on the at least one genomic parameter.

### FIELD OF THE INVENTION

The present invention relates to methods and systems for next-generation sequencing (NGS) of biological molecules. The system can use sequence alignment mapped binary BAM files from user-defined samples as input. Downsampling the mapped BAM files can be used to determine a reduced number of reads needed to obtain critical biological information.

### BACKGROUND OF THE INVENTION

Sequencing costs for biological molecules have decreased about a 100-fold over the past several years to about USD $1000 per genome in 2016 (see, e.g., https://www.genome.gov/27541954/dna-sequencing-costs-data/). However, the need for sequence data and analysis has risen dramatically in recent years because of the ever-expanding number and volume of uses of biological sequence information in medicine, pharmaceutics, diagnostics, as well as a host of new commercial applications. As the number of samples or sequences to be studied increases, the need for efficient storage and analysis of sequence data has greatly increased.

One way to reduce the volume and cost is by multiplexing samples for sequencing. With multiplexing, instead of a single sample being sequenced in a one lane of the sequencer, multiple samples that can be uniquely barcoded are loaded together. The total amount of data that is obtained when samples are multiplexed may be reduced. Unfortunately, in some research applications, relevant biological information can be lost by reducing the total amount of sequence data per sample.

Moreover, it may not be possible to determine or estimate a priori the depth of multiplexing, i.e., the number of samples per lane, required to obtain certain biological information. For example, in some settings, large cohorts can be required for medical studies, clinical trials, drug development, and diagnostic applications. In many cases, data volume can be prohibitive, especially when the sequence data must be stored and analysed repeatedly.

### SUMMARY OF THE INVENTION

It is an object of the present invention to provide a system and method for estimating the depth of sequencing required to gather a sufficient amount of relevant sequencing information in experimental design.

In particular, an object of the present invention is to provide a system and method that solves the above-mentioned problems of the prior art by determining the level of multiplexing and/or the depth of sequencing needed to obtain critical biological information. Deep sequencing on a large number of biological samples can require multiplexing samples to minimize cost of sequencing. In the present invention, the level of multiplexing and depth of sequencing can be determined in advance, so that sequencing data can be obtained without loss of critical biological information. In a sequencing system, a few samples from a pilot study can be sequenced to inform the study design. More specifically, the depth of sequencing can be determined and used for the rest of the samples in a complete study.

According to an exemplary embodiment of the invention, a system and method for sequencing informs the experimental design on the depth of sequencing and thus the level of multiplexing that can be used, while still capturing sufficient biological information. The system requires a small number of pilot samples that are part of the larger experimental design, to be sequenced to determine the effect of any trade-off between biological information and sequencing depth. This system provides the user, e.g., an individual researcher, to perform sequencing at the required depth to obtain complete biological information.

It is contemplated that the above-described objects are to be obtained in a first aspect of the invention by providing a system and method for providing sequencing of biomolecules for differential analysis of a test sample from a normal sample.

In some embodiments, the method can comprise steps for providing a mapped sequence file of each of a pilot test sample and a pilot normal sample, wherein each sequence file has a pilot number of reads; calculating, by a processor, a first test-normal genomic comparison pilot view from the sequence files of the pilot test sample and the pilot normal sample, wherein the first pilot view distinguishes pilot test sample data from pilot normal sample data based on at least one genomic parameter; calculating, by the processor, for each sequence file a downsampled sequence file having a reduced pilot number of reads; calculating, by the processor, a second test-normal genomic comparison pilot view from the downsampled sequence files of the pilot test sample and the pilot normal sample, wherein the second pilot view distinguishes the pilot test sample data from the pilot normal sample data based on the at least one genomic parameter; repeating the downsampling steps for determining the fewest pilot number of reads required for calculating a test-normal genomic comparison view that distinguishes the pilot test sample data from the pilot normal sample data based on the at least one genomic parameter; sequencing biomolecules of the test sample and the normal sample using a number of reads equal to the fewest pilot number of reads; calculating, by the processor, a test-normal genomic comparison view for displaying the differential analysis based on the at least one genomic parameter.

The object of the present invention is solved by the subject matter of the independent claims, wherein embodiments thereof are incorporated in the dependent claims.

### BRIEF DESCRIPTION OF THE DRAWINGS

The methods according to the invention will now be described in more detail with regard to the accompanying figures. The figures showing ways of implementing the present invention and are not to be construed as being limiting to other possible embodiments falling within the scope of the attached claims.

FIG. 1 shows an example of a gene expression distribution for a sample, the initial data having 97 million reads. The data was downsized to 50, 25, 10, 5, 4, 2 and 1 million reads. The analysis shows that as the number of reads decreased, the signal for genes with intermediate transcript abundance levels, e.g., log FPKM (Fragments Per Kilobase Million) values from 1-3, was reduced. The reduced signal can distort the ability to resolve critical biological information. At 4-5 million mapped reads, the distortion becomes significant, and at 1-2 million mapped reads, the distortion prohibits obtaining complete biological information. These data show that at 5 to 10 million mapped reads, the expression profile can be adequately obtained and sequencing coverage is sufficient to reveal complete biological information.

FIG. 2 shows an example of a gene expression distribution for a sample, the initial data having 112 million reads. The data was downsized to 50, 25, 10, 5, 4, 2 and 1 million reads. The analysis shows that as the number of reads decreased, the signal for genes with intermediate transcript abundance levels, e.g., log FPKM (Fragments Per Kilobase Million) values from 1-3, was reduced. The reduced signal can distort the ability to resolve critical biological information. At 4-5 million mapped reads, the distortion becomes significant, and at 1-2 million mapped reads, the distortion prohibits obtaining complete biological information. These data show that at 5 to 10 million mapped reads, the expression profile can be adequately obtained and sequencing coverage is sufficient to reveal complete biological information.

FIG. 3 shows an example of a multi-dimensional scaling plot for sequenced samples, which displays biological information as a difference between the transcriptomes for normal and disease tissue. Each circular point corresponds to a sample, and sample numbers are indicated within the circles. Normal samples are shown in red, and tumour samples are shown in green. The axes are in arbitrary units. Points (samples) appear close together when their transcriptomes are similar. Similarity between transcriptomes can be measured by their Euclidean distance on the plot or by their correlation, such as Spearman, Pearson or Kendall correlation.

FIG. 4 shows an example of a multi-dimensional scaling plot for the sequenced samples in FIG. 3, which were downsampled to 50 million reads.

FIG. 5 shows an example of a multi-dimensional scaling plot for the sequenced samples in FIG. 3, which were downsampled to 1 million reads.

### DETAILED DESCRIPTION OF THE INVENTION

It is an object of the present invention to provide a system and method for altering and determining the sequencing coverage required to obtain pertinent biological information from sequencing data in an experimental design.

More particularly, an object of the present invention is to provide a system and method for determining the level of multiplexing and/or the depth of sequencing needed to obtain critical biological information from samples.

In some embodiments, the optimum level of multiplexing and depth of sequencing can be determined from initial data in advance, so that sequencing data can be obtained at a lower read coverage without loss of critical biological information for additional samples. In a sequencing system, a few samples from a pilot study can be sequenced to determine how biological information can be obtained in the study design. In some cases, the depth of sequencing can be determined and used for the rest of the samples in a complete study.

According to an exemplary embodiment of the invention, a system and method for sequencing informs the experimental design on the coverage of sequencing, and in addition, the level of multiplexing that can be used, while still displaying selected biological information. In some aspects, the system utilizes a small number of pilot samples that are part of the larger experimental design, to be sequenced to determine the effect of any trade-off between biological information and sequencing coverage. This system provides the user, e.g., an individual researcher, to compare the biological information obtainable at different levels of coverage, and then to perform sequencing at a coverage level that provides desired biological information.

It is contemplated that the above-described objects are to be obtained in certain embodiments of the invention by providing a system and method for providing sequencing of biomolecules with downsampling for differential analysis of test samples.

In some embodiments, the method for sequencing biological samples can comprise steps for:

providing mapped sequence files of each of a set of pilot test sample and a set of pilot normal sample, wherein each sequence file has a pilot number of reads;

calculating, by a processor, a first test-normal genomic comparison pilot view from the sequence files of the set of pilot test sample and the set of pilot normal sample, wherein the first pilot view distinguishes pilot test sample data from pilot normal sample data based on at least one genomic parameter;

calculating, by the processor, for each sequence file a downsampled sequence file having a reduced pilot number of reads;

calculating, by the processor, a second test-normal genomic comparison pilot view from the downsampled sequence files of the set of pilot test sample and the set of pilot normal sample, wherein the second pilot view distinguishes the pilot test sample data from the pilot normal sample data based on the at least one genomic parameter;

repeating the downsampling steps for determining the fewest pilot number of reads required for either (1) calculating a test-normal genomic comparison view that sufficiently distinguishes the pilot test sample data from the pilot normal sample data based on the at least one genomic parameter, or (2) generating sample data that shows no or insignificant deviation from the first original sample;

sequencing biomolecules of the test sample and the normal sample using a number of reads equal to the fewest pilot number of reads; and

calculating, by the processor, a test-normal genomic comparison view for displaying the differential analysis based on the at least one genomic parameter.

In addition, another aspect of the present invention is directed to a non-transitory computer readable storage medium for storing one or more programs for sequencing by downsampling, the one or more programs comprising instructions, which when executed by a computing device with a graphical user interface, cause the device to carry out the steps of the method as described above.

The downsampling step can be repeated in an iterative manner, to progressively reduce the number of reads, until the biological information obtained begins to be lost, or degraded, or the resolution of desired features begins to be lost, or degraded.

In some embodiments, a system can use mapped BAM files from user-defined samples as input. New BAM files with lesser number of reads can be created by downsampling the mapped BAM files from user-defined samples.

In some embodiments, the number of reads can be reduced by 50%, or by 60%, or by 70%, or by 80%, or by 90%.

In further embodiments, the number of reads can be reduced by two-fold, or three-fold, or four-fold, or five-fold, or ten-fold.

This method can be repeated for all BAM files from samples that are part of the pilot study.

The system and methods of this invention can be applied to sequencing of whole genomes, exomes, transcriptomes, as well as epigenome sequencing.

Depending on the analyses in which the user is interested, the systems enables evaluation of the simulated down-sampled data. This provides a systematic way for the user to inform his/her decision on sequencing depth necessary to address the pertinent biological question.

The Sequence Alignment/Map (SAM) format can be used for storing large polynucleotide sequence alignments in high-throughput sequencing data. It is a TAB-delimited text format consisting of a header section, which is optional, and an alignment section. BAM is the binary form of SAM.

The SAM format typically includes a header and an alignment section. The binary representation of a SAM file is a BAM file, which is a compressed SAM file. SAM files can be analyzed and edited with the software SAMTOOLS. SAMTOOLS provides various utilities for manipulating alignments in the SAM format, including sorting, merging, indexing and generating alignments in a per-position format. Headings can begin with a “@” symbol, which distinguishes the heading from the alignment section. Alignment sections typically have eleven mandatory fields, and may have a variable number of optional fields. For example, the fields can be QNAME (String) Query template NAME, FLAG (Int) bitwise FLAG, RNAME (String) References sequence NAME, POS (Int) 1-based leftmost mapping POSition, MAPQ (Int) MAPping Quality, CIGAR (String) CIGAR String, RNEXT (String) Reference name of the mate/next read, PNEXT (Int) Position of the mate/next read, TLEN (Int) observed Template LENgth, SEQ (String) segment SEQuence, and QUAL (String) ASCII of Phred-scaled base QUALity+33.

The biological samples of a study may be obtained from cells, organisms, normal tissues, or disease tissues.

According to an exemplary embodiment of the invention, a system and method for sequencing can provide a computed gene expression data for display. In some embodiments, the system and method can detect the level of read coverage, obtained by downsampling, that would be needed to provide certain biological information without an observable and/or significant error, distortion of expression profile, or loss of biological information.

An exemplary system and method utilizes quality metrics for comparing a downsampled or downsized profile against a profile having a larger number of reads, or larger coverage, or greater multiplexing of samples.

In certain embodiments, metrics can be utilized that summarize the difference in expression values across all genes in each sample. Examples of these metrics include root mean square deviation (RMSD), mean/median/percentile absolute deviation, and the like.

In some aspects, metrics can be utilized for characterizing the distortion in the overall gene expression distribution of an individual sample or group of samples. Examples of these metrics include difference in mean, standard deviation, peak, area under histogram, and the like.

In some embodiments, metrics can be utilized that gauge the overall relatedness within (intra) or between (inter) defined groups or clusters of samples. Samples can be grouped according to their nature and characteristics, such as disease subtype or ethnicity, or other clinical trial features, or put into clusters based on computational clustering analysis.

In some embodiments, metrics can be utilized that gauge the overall distance between samples within (intra) or between (inter) defined groups or clusters of samples. Samples can be grouped according to their nature and characteristics, such as disease subtype or ethnicity, or other clinical trial features, or put into clusters based on computational clustering analysis.

In certain aspects, samples of a group can share one or more characteristics that manifest as a certain level of similarity in the expression data, and can be used to distinguish one group from another group. In such embodiments, a metric for degradation of data quality can be a decrease in intra-cluster relatedness and/or an increase in inter-cluster relatedness.

In certain aspects, samples of a group can have one or more characteristics that manifest as a certain level of difference in the expression data, and can be used to distinguish one group member from another member. In such embodiments, a metric for degradation of data quality can be an increase in intra-cluster distance and/or a decrease in inter-cluster distance.

In further embodiments, intra-cluster metrics can be computed by averaging the pairwise comparisons over all combinations of sample pairs from the same cluster. Whereas inter-cluster metrics can be computed by averaging over all combinations of sample pairs with each sample drawn from one of the two different clusters under comparison.

Examples of relatedness metrics as being genomic parameters include correlations, such as Pearson correlation, Spearman correlation, Kendall correlation, and the like.

Examples of distance metrics include Euclidean distance based on the top components of multi-dimensional scaling or principal component analysis.

Metrics can be computed based on the full or specific ranges of gene expression values, or using selected set of genes, e.g. those with higher standard deviations of their gene expressions.

For example, a genomic parameter can be a Spearman's Rank-Order Correlation.

Spearman's rank-order correlation is an example of a nonparametric version of the Pearson product-moment correlation. Spearman's correlation coefficient, ρ, also designated rₛ, can measure the strength and direction of association between two ranked variables. The two variables can be ordinal, interval or ratio. Spearman's correlation can determine the strength and direction of a monotonic association between the two variables, instead of a linear relationship.

Examples of a genomic parameter include linear regression and linear correlation.

To compute whether the quality of sample data is degraded due to downsampling, criteria can be applied that involve one or more of the aforementioned metrics, and on one or multiple gene expression ranges.

In further aspects, downsampling can be done by randomly selecting a fixed number or percentage of reads from the original bulk sequencing data. At each round, data can be processed, for example read alignment and expression quantification, and the resultant gene expression quality evaluated at one or more levels of sequencing coverage. At the coverage level for which the data quality begins to degrade, as compared to data at the next higher level of coverage, and as determined by a set of quality metric criteria, the next round of downsampling can be applied in between the two coverage levels to further the improvement of efficiency. If no degradation in data quality is observed, the next round of downsampling can be applied between zero coverage and the lowest coverage in the current round. This downsampling process can be repeated until: (1) the coverage interval is small enough, bringing little or no further impact on sequencing efficiency, when searching for a lower optimum coverage, or (2) the improvement in data quality becomes negligible or the data quality is sufficiently high when searching for the minimum coverage that can satisfy the data quality requirements.

In some aspects, the system and methods of this invention can be used to measure the expression levels of all genes over a wide dynamic range without loss of sensitivity, and/or without introducing measurement noise or errors.

According to exemplary embodiments of this invention, the lower bound for sequencing coverage that is needed for detecting a gene expression profile of a sample without distortion or loss of information can be identified. The lower bound for sequencing coverage can be used to acquire and/or process additional data for a larger study, thereby greatly increasing efficiency, reduce the sequencing data storage and processing effort, and improving the quality of diagnostic tests that utilize the sequencing results.

### Example 1

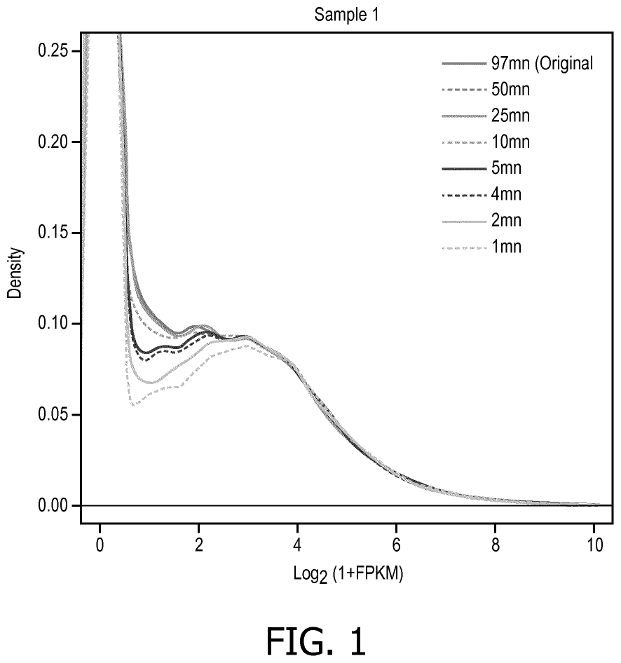

FIG. 1 shows an example of a gene expression distribution for a sample, the initial data having 97 million reads. The data was downsized to 50, 25, 10, 5, 4, 2 and 1 million reads. The analysis shows that as the number of reads decreased, the signal for genes with intermediate transcript abundance levels, e.g., log FPKM (Fragments Per Kilobase Million) values from 1-3, was reduced. The reduced signal can distort the ability to resolve critical biological information. At 4-5 million mapped reads, the distortion becomes significant, and at 1-2 million mapped reads, the distortion prohibits obtaining complete biological information. These data show that at an advantageously low level of 5 to 10 million mapped reads, the expression profile was adequately obtained and sequencing coverage was sufficient to reveal complete biological information.

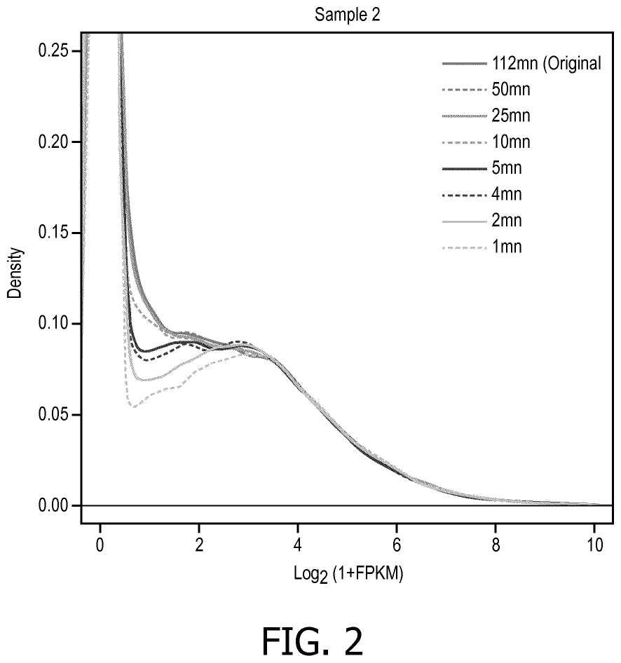

FIG. 2 shows an example of a gene expression distribution for a sample, the initial data having 112 million reads. The data was downsized to 50, 25, 10, 5, 4, 2 and 1 million reads. The analysis shows that as the number of reads decreased, the signal for genes with intermediate transcript abundance levels, e.g., log FPKM (Fragments Per Kilobase Million) values from 1-3, was reduced. The reduced signal can distort the ability to resolve critical biological information. At 4-5 million mapped reads, the distortion becomes significant, and at 1-2 million mapped reads, the distortion prohibits obtaining complete biological information. These data show that at an advantageously low level of 5 to 10 million mapped reads, the expression profile was adequately obtained and sequencing coverage was sufficient to reveal complete biological information.

### Example 2

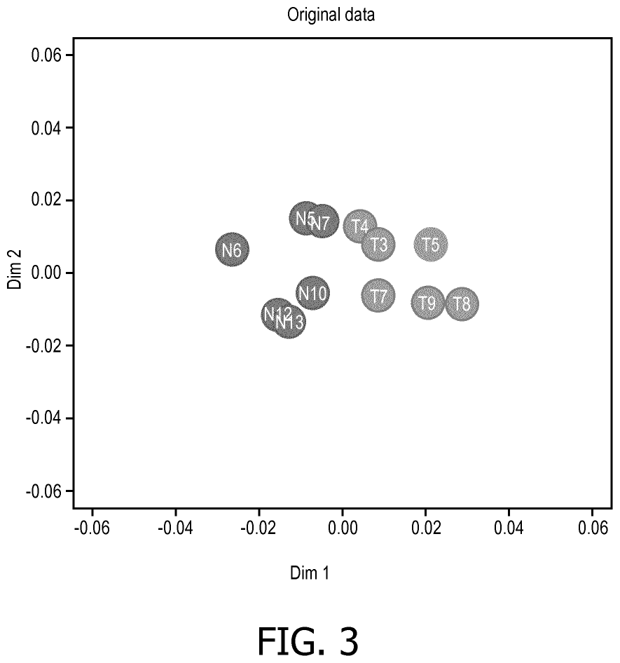

FIG. 3 shows an example of a multi-dimensional scaling plot for sequenced samples, which displays biological information as a difference between the transcriptomes for normal and disease tissue. Each circular point corresponds to a sample, and sample numbers are indicated within the circles. Normal samples are shown in red, and tumour samples are shown in green. The axes are in arbitrary units. Points (samples) appear close together when their transcriptomes are similar. Similarity between transcriptomes can be measured by their Euclidean distance on the plot or by their correlation, such as Spearman, Pearson or Kendall correlation. FIG. 3 was calculated from the RNA-seq data of Boj et al., Organoid Models of Human and Mouse Ductal Pancreatic Cancer, Cell Vol. 160, pp. 324-338, Jan. 15, 2015.

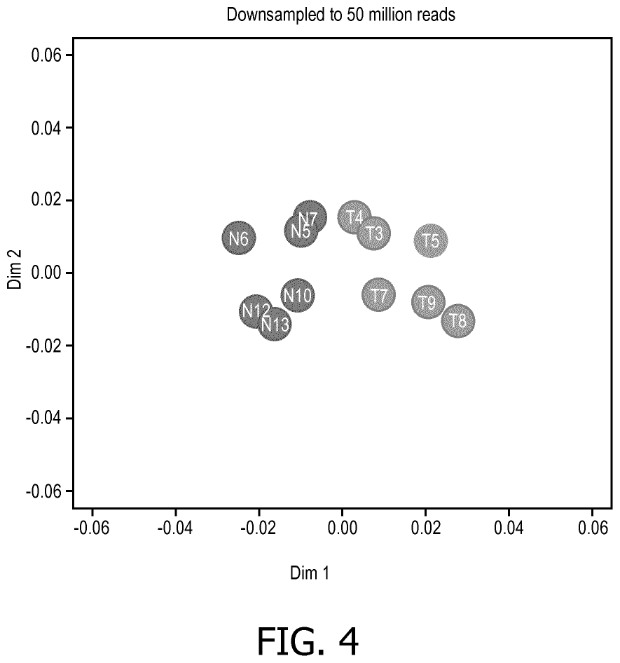

FIG. 4 shows an example of a multi-dimensional scaling plot for the sequenced samples in FIG. 3, which were downsampled to 50 million reads.

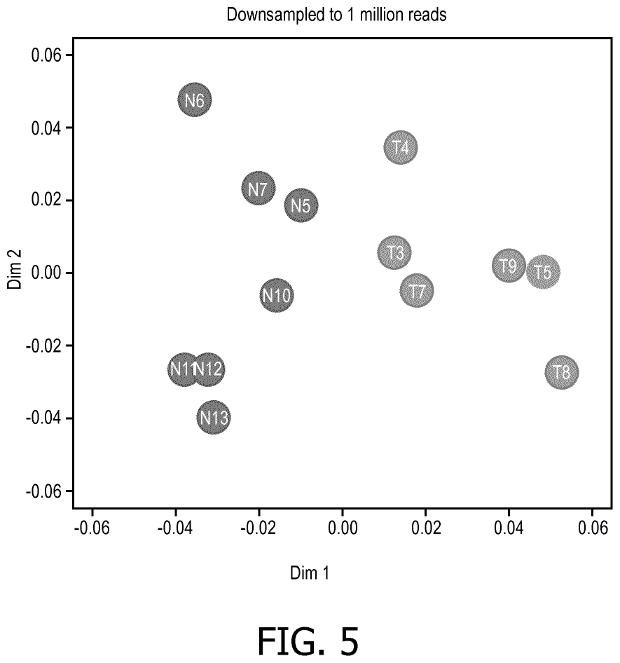

FIG. 5 shows an example of a multi-dimensional scaling plot for the sequenced samples in FIG. 3, which were downsampled to 1 million reads. Surprisingly, distinct differences in the overall spatial arrangement of the samples were revealed for this low number of reads, even comparable to data requiring 50-fold to 100-fold greater size. The main differences between the tumor and normal transcriptomes were clearly visible, even at a surprisingly low sequencing level of 1 million reads. Thus, the required sequencing depth was greatly reduced, providing an unexpectedly advantageous ability to distinguish tumor from normal samples.

All publications, references, patents, patent publications and patent applications cited herein are each hereby specifically incorporated by reference in their entirety for all purposes.

While certain embodiments, aspects, or variations have been described, and many details have been set forth for purposes of illustration, it will be apparent to those skilled in the art that additional embodiments, aspects, or variations may be contemplated, and that some of the details described herein may be varied considerably without departing from what is described herein. Thus, additional embodiments, aspects, and variations, and any modifications and equivalents thereof which are understood, implied, or otherwise contemplated are considered to be part of the invention(s) described herein. For example, the present application contemplates any combination of the features, terms, or elements of the various illustrative components and examples described herein.

The use herein of the terms “a,” “an,” “the” and similar terms in describing the invention, and in the claims, are to be construed to include both the singular and the plural, for example, as “one or more.”

The terms “comprising,” “having,” “include,” “including” and “containing” are to be construed as open-ended terms which mean, for example, “including, but not limited to.” Thus, terms such as “comprising,” “having,” “include,” “including” and “containing” are to be construed as being inclusive, not exclusive.

The examples given herein, and the exemplary language used herein are solely for the purpose of illustration, and are not intended to limit the scope of the invention. All examples and lists of examples are understood to be non-limiting.

1. A method for sequencing biomolecules for differential analysis of a test sample from a normal sample, the method comprising:
providing a mapped sequence file of each of a pilot test sample and a pilot normal sample, wherein each sequence file has a pilot number of reads;
calculating, by a processor, a first test-normal genomic comparison pilot view from the sequence files of the pilot test sample and the pilot normal sample, wherein the first pilot view distinguishes pilot test sample data from pilot normal sample data based on at least one genomic parameter;
calculating, by the processor, for each sequence file a downsampled sequence file having a reduced pilot number of reads;
calculating, by the processor, a second test-normal genomic comparison pilot view from the downsampled sequence files of the pilot test sample and the pilot normal sample, wherein the second pilot view distinguishes the pilot test sample data from the pilot normal sample data based on the at least one genomic parameter;
repeating the downsampling steps for determining the fewest pilot number of reads required for calculating a test-normal genomic comparison view that distinguishes the pilot test sample data from the pilot normal sample data based on the at least one genomic parameter;
sequencing biomolecules of the test sample and the normal sample using a number of reads equal to the fewest pilot number of reads; and
calculating, by the processor, a test-normal genomic comparison view for displaying the differential analysis based on the at least one genomic parameter.

2. The method of claim 1, wherein the mapped sequence files are BAM files or SAM files.

3. The method of claim 1, wherein the biomolecules are polynucleotides or polypeptides.

4. The method of claim 1, wherein the biomolecules are DNA, RNA, or protein.

5. The method of claim 1, wherein the differential analysis distinguishes a disease test sample from a normal sample.

6. The method of claim 1, wherein the differential analysis distinguishes a tumor test sample from a normal sample.

7. The method of claim 1, wherein the pilot number of reads is reduced to 5 million.

8. The method of claim 1, wherein the pilot number of reads is reduced to 1 million.

9. The method of claim 1, wherein the number of reads equal to the fewest pilot number of reads is 5 million.

10. The method of claim 1, wherein the number of reads equal to the fewest pilot number of reads is 1 million.

11. The method of claim 1, wherein the mapped BAM files are supplied by next generation sequencing.

12. The method of claim 1, wherein the sequencing of the biomolecules is performed by multiplexing samples.

13. The method of claim 1, wherein the test-normal genomic comparison view displays relative gene expression levels.

14. The method of claim 1, wherein the number of reads equal to the fewest pilot number of reads is sufficient to distinguish an expression level of a test sample from a normal sample.

15. The method of claim 1, wherein the number of reads equal to the fewest pilot number of reads is sufficient to determine expression levels of a test sample from a normal sample for all genes over a wide dynamic range without loss of sensitivity.

16. The method of claim 1, wherein the test-normal genomic comparison view displays transcriptome clusters.

17. The method of claim 1, wherein the test-normal genomic comparison view displays transcriptome clusters that are distinguished by Spearman's correlation coefficient, a Pearson's correlation coefficient, or a Kendall's correlation coefficient.

18. The method of claim 1, wherein the test sample is a disease cell or disease tissue sample.

19. The method of claim 1, wherein the test sample is a model cell or model tissue sample.

20. The method of claim 1, wherein the test sample is a human sample or animal sample.

21. A non-transitory computer readable medium carrying software instructions configured to perform the steps of:
receiving and storing a mapped sequence file of each of a pilot test sample and a pilot normal sample, wherein each sequence file has a pilot number of reads;
calculating, by a processor, a first test-normal genomic comparison pilot view from the sequence files of the pilot test sample and the pilot normal sample, wherein the first pilot view distinguishes pilot test sample data from pilot normal sample data based on at least one genomic parameter;
calculating, by the processor, for each sequence file a downsampled sequence file having a reduced pilot number of reads;
calculating, by the processor, a second test-normal genomic comparison pilot view from the downsampled sequence files of the pilot test sample and the pilot normal sample, wherein the second pilot view distinguishes the pilot test sample data from the pilot normal sample data based on the at least one genomic parameter;
repeating the downsampling steps for determining the fewest pilot number of reads required for calculating a test-normal genomic comparison view that distinguishes the pilot test sample data from the pilot normal sample data based on the at least one genomic parameter;
sequencing biomolecules of the test sample and the normal sample using a number of reads equal to the fewest pilot number of reads; and
calculating, by the processor, a test-normal genomic comparison view for displaying the differential analysis based on the at least one genomic parameter.
